(19) United States
(12) Patent Application Publication
(10) Pub. No.: US 2016/0287226 A1
(43) Pub. Date: Oct. 6, 2016

(54) APPARATUS FOR CLOSURE OF ATRIAL SEPTAL DEFECTS

(71) Applicants: MUSTAFA H. ABDULLAH AL-OBANDI, SAFAT (KW); ERAN LEVIT, CONCORD, NH (US)

(72) Inventors: MUSTAFA H. ABDULLAH AL-OBANDI, SAFAT (KW); ERAN LEVIT, CONCORD, NH (US)

(21) Appl. No.: 14/677,947

(22) Filed: Apr. 2, 2015

(51) Int. Cl. A61B 17/00; A61B 19/00

(52) U.S. Cl. CPC A61B 17/0057; A61B 17/00234; A61B 19/54; A61B 2017/00575; A61B 2017/00606; A61B 2017/00243; A61B 2017/003; A61B 2019/5466

(57) ABSTRACT

The apparatus for closure of atrial septal defects includes a sheath having a proximal portion and a distal portion, the sheath defining a lumen extending therethrough, a handle positioned in communicating relation with the proximal portion of the sheath, and a balloon positioned in communicating relation the distal portion of the sheath. The distal portion of the sheath can include a soft tip having at least one hole defined therein. The handle can have a bidirectional control configured for controlling or deflecting the direction of the distal portion of the sheath by at least 90 degrees to aid in positioning and aligning the balloon, as well as an occlusion device into the ASD. The balloon, such as a sizing balloon, can measure the size of the ASD within the interatrial septum of a heart of a patient.

### BACKGROUND OF THE INVENTION

1. Field of the Invention

The present invention relates generally to medical devices, and, more particularly, to an apparatus that permits measuring the size of an atrial septal defect (ASD) and placement of an occlusion device to repair the ASD.

2. Description of the Related Art

In a normal functioning heart, the left atrium receives the oxygenated blood from the pulmonary veins and pumps the blood into the left ventricle, which pumps the oxygen-rich blood to the brain, organs, and tissues of the body. The right atrium receives the deoxygenated blood from the superior and inferior vena cava and other cardiac veins and pumps it into the right ventricle, which pumps the deoxygenated blood into the pulmonary system to replenish its oxygen supply. Normally, the left atrium and the right atrium are separated by a septum known as the interatrial septum that prevents the oxygen-rich blood in the left atrium from mixing with the deoxygenated blood in the right atrium.

If the interatrial septum fails to properly develop, however, an atrial septal defect (ASD) can result. An ASD is a hole in the interatrial septum that enables blood flow to cross between the left atria and the right atria, or vice versa, allowing the oxygen-rich blood to mix with the deoxygenated blood. If the shunt is left untreated, it can lead to lower than normal oxygen in the atrial blood that is pumped from the left atrium to the brain, organs, and tissues of the body, which can eventually lead to the development of a cardiac arrhythmia, decompression sickness, Eisenmenger's syndrome, paradoxical embolus, and even migraines.

There are various methods and apparatuses used for closing ASDs. Typically, surgery, such as open heart surgery, as well as interventional occlusion devices can be utilized to close ASDs. It is to be noted that utilizing a percutaneous closure, such as an interventional occlusion device, to close an ASD can result in a faster and easier recovery for a patient, since the percutaneous closure is minimally invasive and only requires the passage of a catheter into the heart through a femoral vein instead of surgery.

Typically, the approach utilized for closing an ASD with an interventional occlusion device requires the use of three steps. For example, a medical practitioner first threads a guiding catheter (the first catheter) through a percutaneous femoral vein into the heart and then into the ASD. Once the guiding catheter is positioned in the ASD, a wire is advanced into the catheter. The wire is left in place and the first catheter is removed. A second catheter having a sizing or measuring balloon is passed over the wire and into the ASD, where the sizing or measuring balloon is inflated to determine the size of the ASD. Once the size of the ASD is determined, the second catheter is removed over the wire and a third catheter (a sheath with a dilator) is passed over the same wire to the left atrium. The wire and the dilator are removed and a properly sized occlusion device is passed into the sheath and positioned in the ASD so as to deploy the occlusion device.

A typical occlusion-type device includes two discs, one that is positioned at the distal side of the interatrial septum and the other positioned at the proximal side of the interatrial septum. Ideally, once the second disc is introduced, the two discs are secured to one another by means of short spring arms or resilient wires traversing the ASD, thereby forming a “sandwich” to cover the ASD. Once the device has been properly aligned and positioned over the ASD, the cable is separated from the device. At this stage, tissue can begin to form over the device so as to completely close the ASD.

The positioning and alignment of the occlusion device can be difficult and time consuming, since various catheters, such as a guiding catheter, a second catheter having a sizing or measuring balloon, and a third catheter having the occlusion device, are required to deploy a properly sized occlusion device into the ASD. If both discs of the occlusion device are not properly positioned and aligned within ASD, the “sandwich” will not properly cover the ASD and blood can continue to flow between the right and left atria. Another issue that can arise is the proper measurement of the ASD and its edges, since the respective dimensions of an ASD can vary from one patient to another. If the edge around the ASD is small or deficient, the occlusion device may not open completely and/or align properly and may become oblique across the ASD.

Thus, an apparatus for closure of atrial septal defects solving the aforementioned problems is desired.

### SUMMARY OF THE INVENTION

An embodiment of an apparatus for closure of atrial septal defects can include a sheath having a proximal portion and a distal portion, the sheath defining a lumen extending therethrough, a handle positioned in communicating relation with the proximal portion of the sheath, and a balloon positioned in communicating relation with the distal portion of the sheath. The distal portion of the sheath can include a soft tip having at least one hole defined therein. Further, the handle can have a bidirectional control configured for controlling or deflecting the direction of the distal portion of the sheath by at least 90 degrees to aid in positioning and aligning the balloon and an occlusion device within an ASD. The balloon, such as a sizing balloon, can measure the size of the ASD within the interatrial septum of a heart of a patient. After the size of the ASD has been determined, a medical practitioner can thread a properly sized device (which may be an amplatzer device) through the same sheath and into the ASD. Once through the ASD, the first disc is opened and pulled against the distal side of the interatrial septum and the other disc is opened and positioned at the proximal side of the interatrial septum so as to occlude the ASD. This technique can convert the three-step process initially described into a two-step process or, even, a single step procedure.

These and other features of the present invention will become readily apparent upon further review of the following specification and drawings.

### BRIEF DESCRIPTION OF THE DRAWINGS

FIG. 1A is a perspective view of an embodiment of an apparatus for closure of atrial septal defects according to the present invention.

FIG. 1B is an partial, exploded view of an embodiment of a distal portion of an apparatus for closure of atrial septal defects according to the present invention.

FIG. 2 is a side, cross sectional view of an embodiment of a handle for an apparatus for closure of atrial septal defects according to the present invention.

FIG. 3A is a partial view of an embodiment of a sheath of an apparatus for closure of atrial septal defects in which the distal portion of the sheath is deflected to the right according to the present invention.

FIG. 3B is a partial view of an embodiment of a sheath of an apparatus for closure of atrial septal defects in which the distal portion of the sheath is deflected to the left according to the present invention.

FIG. 4 is an environmental view in section of an occlusion device being implanted into an atrial septal defect using an apparatus of the prior art.

FIG. 5 is an environmental view in section showing use of an apparatus for closure of atrial septal defects according to the present invention having a sizing balloon to size the defect and with an occlusion device having an opened left atrial disc being implanted into the atrial septal defect.

Similar reference characters denote corresponding features consistently throughout the attached drawings.

### DETAILED DESCRIPTION OF THE PREFERRED EMBODIMENTS

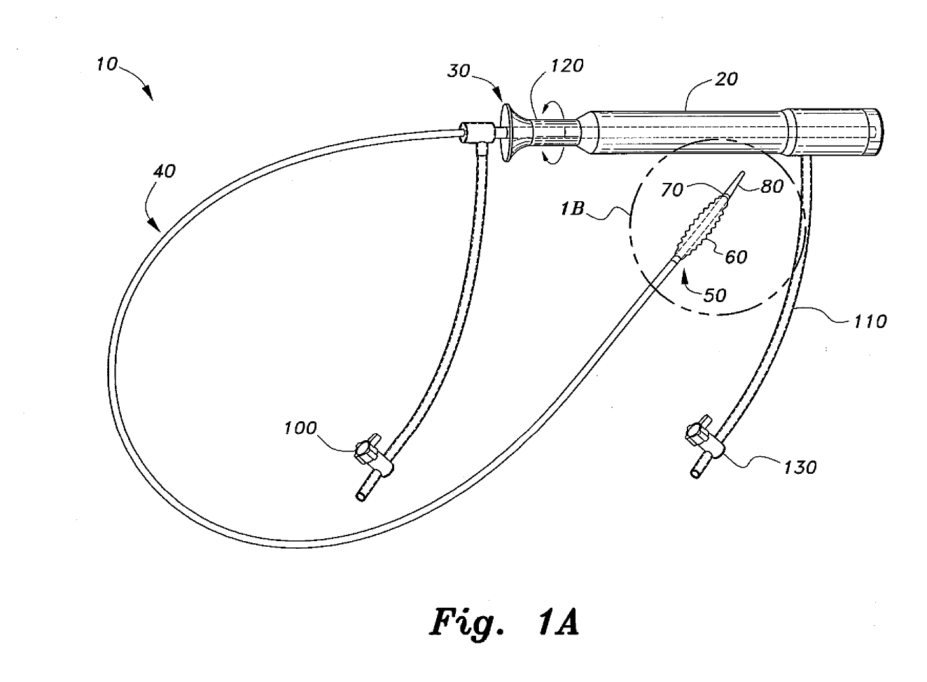

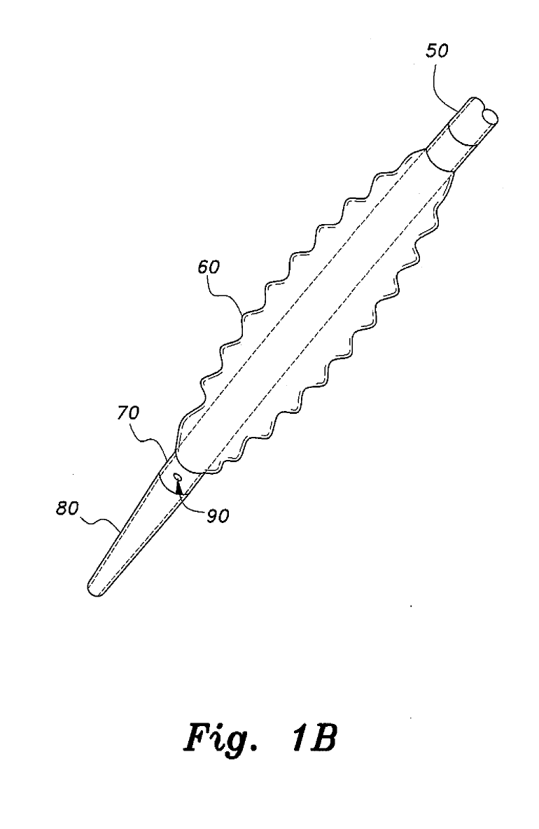

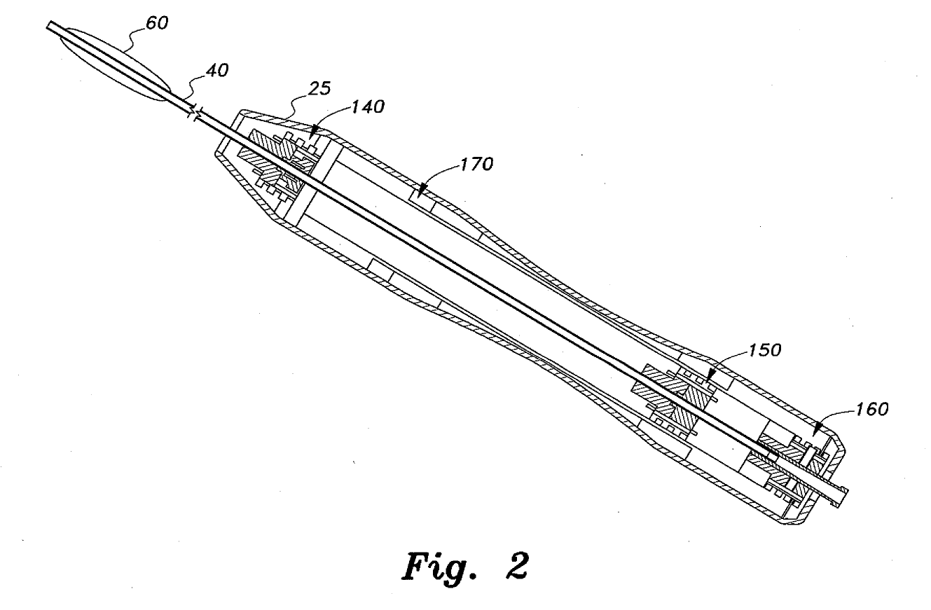

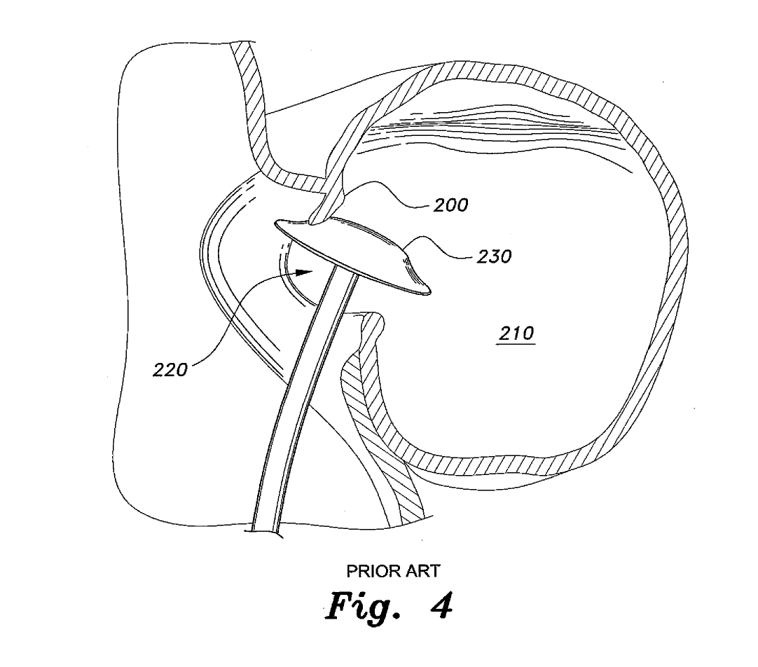

Referring to FIGS. 1A-5, an apparatus 10 for closure of atrial septal defects is generally illustrated. The apparatus 10 includes a sheath 40 having a proximal portion 30, such as a flexible proximal portion, and a distal portion 50, the sheath 40 defining a lumen extending therethrough, the sheath 40 being configured for receiving a medical implement 300, a balloon 60 positioned on the distal portion 50 of the sheath 40, wherein the balloon 60 is configured for measuring the size of an ASD 220 within an interatrial septum 200 of a patient, and a handle 20 configured for positioning and aligning the balloon 60 and the medical implement 300 within the ASD 220, the handle 20 being positioned in communicating relation with the proximal portion 30 of the sheath 40.

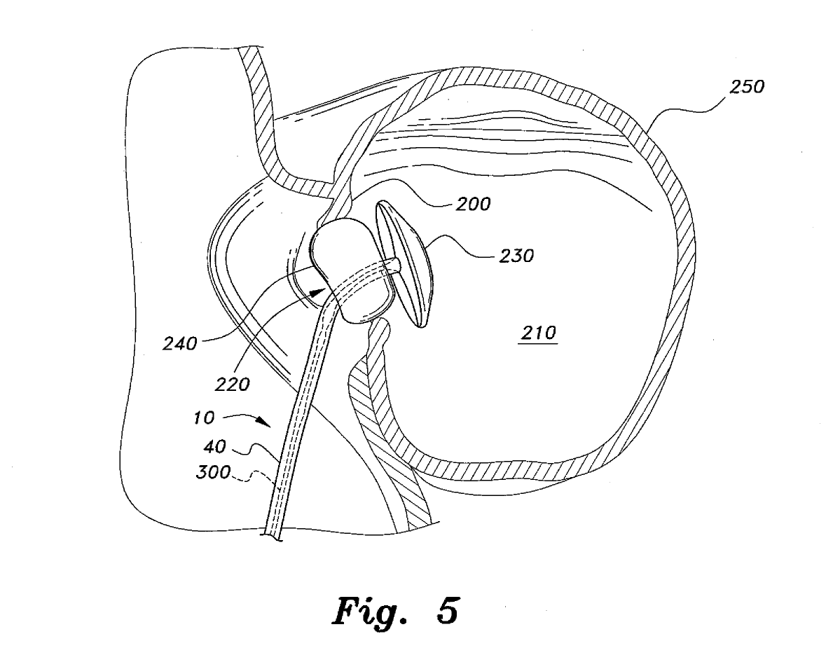

The sheath 40 can be adapted for placement within the ASD 220 in the interatrial septum 200 of the patient. The sheath 40 can include a wall of substantially uniform thickness having an outer surface and an inner surface configured for receiving and allowing the medical implant 300, such as a catheter, having an occlusion device 230, to pass therethrough and into the interatrial septum 200 of the patient so as to repair the ASD 220 (as illustrated in FIG. 5). The sheath 40, such as an elongate, flexible, braided sheath, can be formed from any type of medical grade material, and is preferably an elongate, flexible tubular structure having a braided construction. The braided construction of the sheath 40 can enhance torqueability, pushability, and kink resistance when threading the sheath 40 through a blood vessel, such as the femoral vein. The braided portion of the sheath 40 can be formed from an outside metal braid, a polytetrafluoroethylene (PTFE) core, and a Pebax® cover. While the sheath 40 can vary in length it is desirable that the sheath 40 have a usable length of 81 cm. Further, the sheath 40 can also have a diameter of any suitable French (Fr) size, such as 8.5 Fr, 10.5 Fr, or 12.5Fr.

The distal portion 50 of the sheath 40 can include a soft tip 70, such as a soft, arcuate tip, having at least one hole 90 (FIG. 1B) defined therein. The distal portion 50 of the sheath 40 can also include a plurality of flexible segments with varying degrees of stiffness, as well as can have a pre-shaped curve, preferably an obtuse curve. Further, the distal portion 50 can include a steerable distal portion such that as the sheath 40 is being threaded through the femoral vein and into the heart 250, a medical practitioner can steer the distal portion 50 of the sheath 40 into the ASD 220 within the interatrial septum 200 of the patient. It is to be appreciated that a dilator tip 80 can be positioned in communicating relation with the soft tip 70 at the distal portion 50 of the sheath 40 so as to aid in threading the sheath 40 into the ASD 220.

The soft tip 70 positioned on the distal portion 50 of the sheath 40 can eliminate any undue harm or injury to the skin, tissue, and vessel. The soft tip 70 portion of the sheath 40 can be formed from soft Pebax®, in contrast to the dilator 80, which can be formed from hard Pebax®. It is to be noted that the distal portion 50 can contain a radiopaque tip marker to aid in determining proper placement of the sheath 40 and the balloon 60 during a procedure. The radiopaque tip marker can be formed from at least one material selected from a group consisting of barium sulfate, bismuth subcarbonate, bismuth oxychloride, bismuth trioxide, and tungsten. The distal portion 50 of the sheath 40 can also include a second marker spaced in a defined distance from the first marker to serve as a sizing marker. The proximal portion 30 of the sheath 40, on the other hand, can be adapted to include distinctive indicia, such as color-coding, markings, and/or etchings along the entire length of the sheath 40 to indicate orientation and depth of penetration into the vessel, such as the femoral vein.

The balloon 60, such as a sizing balloon, coupled to the distal portion 50 of the sheath 40 can be formed from any suitable, strong, puncture-resistant medical grade material, such as polyethylene terephthalate (PET), nylon, polyurethane, and other elastomers. Further, the balloon 60 can be attached to the sheath 40 by any type of suitable adhesive, such as UV adhesive or Loctite®4011. The balloon 60 can also be adapted to expand to a specific size, such as the size of the ASD 220, The balloon 60 can be produced in a wide range of diameters, lengths, and shapes, such as conical, spherical, square, dog-bone, stepped, tapered, and offset. It is desirable, however, that the balloon 60 have a length of about 3-4 cm and a spherical shape adapted to determine the correct size of the ASD 220. The balloon 60 can also be coated for lubrication or for abrasion resistance. Further, it is to be noted that the apparatus 10 can include a balloon inflation port 100, such as extending from the sheath 40 adjacent to the handle 20, so as to inflate the balloon 60.

The handle 20 can include a handle cover 25, as well as any suitable shape, such as an ergonomic shape having a curved diameter and a French (Fr) size indicator. It is to be noted that the handle 20 can include additional components, such as a sheath flush port 130 connected to the handle 20 by tubing 110, which can be used to flush the apparatus 10 with saline.

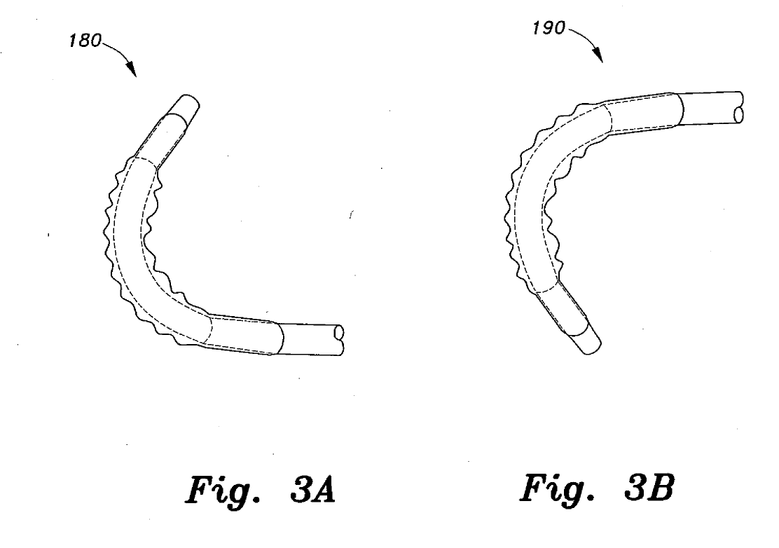

The handle 20, can include a bi-directional control 120, such as bi-directional control rotatably mounted thereon. The bi-directional control 120 can be configured for controlling or deflecting the direction of the distal portion 50 of the sheath 40 by at least 90 degrees to aid in positioning and aligning the balloon 60, as well as the occlusion device 225, within the ASD, as illustrated in FIGS. 3A and 3B. For example, the bi-directional control 120 can deflect the soft tip 70 positioned on the distal portion 50 of the sheath 40 according to a compound curve to position the balloon 60 in an area of the human body, such as within the ASD 220 of the interatrial septum 200 of the patient. The bi-directional control 120 can deflect the soft tip 70 towards the target location through a minimally invasive approach, such as through a patient's vasculature, and can provide for manipulation of the distal portion 50 of the sheath 40 at the target location, such as to approximate a septal wall. It is to be noted that the handle 20 can include a plurality of steerable handle drivers, such as a first steerable handle driver 140, a second steerable handle driver 150, and a third steerable driver 160, as well as at least one articulating handle driver 170, which can be used in connection with the bi-directional control 120.

The bi-directional control 120 can include any type of suitable steering mechanism commonly known in the art, such as the steering mechanisms disclosed in U.S. Pat. No. 7,666,204, U.S. Pat. No. 7,226,467, and U.S. Pat. No. 8,323,239, which are hereby incorporated by reference. For example, the sheath 40 can be steered by using two pull wires, each pull wire can be disposed at least 180 degrees apart and positioned along the circumference of the sheath 40. Further, each pull wire can be funneled through a dedicated lumen and can be positioned in the interior of the sheath 40. The pull wires can extend along the sheath 40 from the proximal portion 30 through to the distal portion 50. It is to be noted that the two pull wires extending along the interior of the sheath 40 can connect the bi-directional control 120 in the handle 20 with the distal end 50 of sheath 40. The two pull wires can be coupled to the bi-directional control 120 by any suitable adhesive, such as UV adhesive, so as to allow the distal portion 50 of the apparatus 10 to deflect when the bi-directional control 120 is rotated about the axel of the handle 20.

It is to be noted that the rotational movement of the bi-directional control 120 translates using a “screw” mechanism into an axial displacement so as to pull the two pull wires. For example, as tension is applied to the end of one pull wire positioned in the proximal portion 30 of the sheath 40, the tension travels along the pull wire towards the end of the pull wire positioned in the distal portion 50 of the sheath 20. The tension can then shorten one side of the distal portion 50 of the sheath 40, such as a buckling mechanism, and can cause that side to deflect in a particular direction. For example, the bi-directional control 120 may be used to steer the distal portion 50 of the sheath 40 through a 180° deflection to dispose the distal portion 50 of the sheath 40 to the right or to the left, as illustrated in FIGS. 3A and 3B, as well as in an upward or downward direction, in order to position the sheath 40 in communicating relation to the ASD 220, such as in the left atrium.

By way of operation, a medical practitioner can insert an introducer (not shown) into a patient's groin area to reach a vessel, such as the femoral vein, so that the practitioner may insert a guide wire (not shown) into the vessel and thread the sheath 40 along the guide wire (not shown) into the patient's heart 250 and into the ASD 220 within the interatrial septum 200. The dilator 80 coupled to the sheath 40, can have a tip that can be tapered so that the tip of the dilator 80 can smoothly follow the guide wire (not shown) into the femoral vein without causing any undue harm or injury to the internal walls of the patient's blood vessel.

Once the sheath 40 is properly positioned and aligned within the ASD 220, the balloon 60 (FIGS. 1a, 1b, and 5) can be inflated by use of the balloon inflation port 100 to form an inflated balloon 240. The balloon 60 can also be inflated using dye diluted in saline to enable the medical practitioner to see the inflated balloon 240 by both echocardiography and fluoroscopy. The inflated balloon 240 can then be used to measure the size of the ASD 220 in the interatrial septum 200 of the patient, as illustrated in FIG. 5.

After the size of the ASD 220 has been determined, but without removing the sheath 40 from the ASD 220 within the patient's interatrial septum 200, the medical practitioner can thread the medical implement 300 having the occlusion device 230, which may be a disc-type occlusion device, a clamshell-type occlusion device, or a Watchman® occlusion device, through the most proximal part of the apparatus 10 into the left atria 210 of a heart 250. Upon entering the left atria 210 of the heart, the occlusion device 230 can be deployed and expanded so as to cover a side of the ASD 220. The occlusion device 230 can be held in position by the inflatable balloon 240 coupled to the distal portion 50 of the sheath 40. For example, the inflated balloon 240 can prevent the occlusion device 230 from sliding out of the ASD 220. Once the occlusion device 230 is properly aligned and positioned over the ASD 220, the medical implement 300 is detached from the occlusion device 230, which provides an area for tissue to grow and cover the ASD 220.

If the medical practitioner determines that the ASD 220 is difficult to close due to deficient rims, then the curvature (illustrated in FIG. 3A and 3B) of the distal portion 50 of the sheath 40 can be used to properly position and align the occlusion device 230 within the ASD 220 and close it successfully, as illustrated in FIG. 5. It is to be understood that the apparatus 10 can also be used in conjunction with stents or other implantation devices requiring a measuring tool together with the stent or other such device.

It is to be understood that the present invention is not limited to the embodiments described above, but encompasses any and all embodiments within the scope of the following claims.

1. An apparatus for closure of atrial septal defects, comprising:
a handle, the handle having a bidirectional control rotatably mounted thereon;
a braided, flexible sheath in communication with the handle, the sheath having a proximal portion and a distal portion and having a length of 81 cm, the proximal end of the sheath being attached to the bidirectional control for rotation therewith, the sheath defining a lumen extending therethrough, the distal portion having a soft tip portion having at least one hole defined therein and a dilator at the terminal end of the tip portion wherein the dilator is harder than the soft tip portion, further wherein the sheath has an 8.5-12.5 Fr size;
an occlusion device, the occlusion device being sized for passage through the sheath and into the interatrial septum of the patient to repair the atrial septal defect; and
a balloon positioned in communicating relation with the distal portion of the sheath, the balloon configured for measuring the size of an atrial septal defect within an interatrial septum of a patient and for positioning and aligning a medical implement having the occlusion device within the interatrial septum of the patient to repair the atrial septal defect.

2-3. (canceled)

4. The apparatus for closure of atrial septal defects according to claim 1, further comprising a sheath flush port connected to the handle.

5-6. (canceled)

7. The apparatus for closure of atrial septal defects according to claim 1, further comprising a radiopaque tip marker disposed on the distal portion of the sheath.

8. The apparatus for closure of atrial septal defects according to claim 1, further comprising a balloon inflation port extending from the sheath adjacent said handle and tubing extending from the balloon inflation port through the sheath to said balloon.

9. (canceled)
